(19) United States
(12) Patent Application Publication
(10) Pub. No.: US 2006/0282164 A1
(43) Pub. Date: Dec. 14, 2006

(54) Implant shell and filler apparatus

(76) Inventor: Joann Seastrom, Chicago, IL (US)

Correspondence Address: Lesavich High-Tech Law Group, P.C.;Suite 325, 39 S. LaSalle Street, Chicago, IL, 60603, US

(21) Appl. No.: 11/378,818

(22) Filed: Mar. 17, 2006

Related U.S. Application Data
(60) Provisional application No. 60/688,576, filed on Jun. 8, 2005.

(51) Int. Cl. A61F 2/12; A61F 2/02

(52) U.S. Cl. 623/8; 623/23.74

(57) ABSTRACT

An implant shell and filler apparatus. The implant shell includes a pre-determined surface material with a microbead filler. The implant she apparatus create a viable matrix for tissue growth that can also enhance the look, feel and touch of implants used in humans and animals. The implant shell and filler apparatus are made of an inert biocompatible material with non-permeable, permeable and/or semi-permeable characteristics. The implant shell and filler apparatus replaces, augments or alters human breast or human testicle tissue or provides other human tissue contouring. The implant shell and filler apparatus may also be used for animal tissues.

### CROSS REFERENCES TO RELATED APPLICATIONS

This application claims priority to U.S. Provisional Application No. 60/688,576, filed Jun. 8, 2005, the contents of which are incorporated by reference.

### FIELD OF THE INVENTION

This invention relates to human and animal implants. More specifically, it relates to an implant shell and filler apparatus.

### BACKGROUND OF THE INVENTION

Each year in the United States and other countries, there are a number of women and men who develop breast cancer and require removal of breast tissue as part of a treatment plan for the breast cancer. Such men and woman often opt for breast reconstruction surgery that includes the use of breast implants. There are also a number of women and an increasing number of men who desire to change the size of their own breast tissues to make them larger or smaller. In 2005, about eighty percent of all breast implants in the United States were for cosmetic reasons and about twenty percent for breast reconstruction after breast cancer surgery.

Prior to the 1960's, methods of breast enlargement and replacement included paraffin injections, silicone injections, the insertion of sponges and other materials. None of these methods achieved satisfactory long-term results. In addition, autoimmune reactions to these materials and injections proved to be extremely dangerous to the health of the receipient.

In 1963, the first silicone gel-filled breast implants were introduced, followed by the introduction of saline-filled implants in 1965. Such breast implants are soft silicone sacs or shells, inflated with either saline solution (i.e., salt water) or a synthetic silicone gel. From the 1960's until the early 1990's, about ninety-five percent of all breast implants included sacs filled with silicone gel because they had a more pleasing and natural look and feel than the saline-filled implants.

In the 1970's, inflatable breast implants were introduced. In 1976, double lumen implants appeared on the market. Double lumen implants had an interior chamber filled with silicone gel and an outer, saline-filled chamber.

Silicone was initially assumed to be biologically inert and have no harmful biological effects. However, cases of connective tissue and autoimmune disorders related to breast implants filled with silicone gels began to be reported. In 1992, the Food and Drug Administration (FDA) banned breast implants filled with silicone gels except those used in specific and authorized clinical studies. Since the 1992 FDA ban, most breast implants have been saline-filled. The FDA has recently allowed the use of silicone breast implants once again for selected groups of patients.

In the late 1980s, textured-surface silicone and saline breast implants were introduced on the theory that a textured surface would modify the process of scar formation and reduce the incidences of increased breast hardness (i.e., capsular contracture or encapsulation) caused by scar tissue contracting around the implant. The results of using such textured-surface silicone and saline breast implants have been mixed. Incidences of capsular contracture (i.e., increased breast hardening, etc. caused by scar tissue contracting around the implant) are still one of the most common problems associated with breast implants.

There are many problems associated with using silicone and saline breast implants. One problem is that breast implants using saline filler result in implants that are harder, less pliable than a natural body part. Implants using silicone gel filler result in a more natural feel but, if the shell ruptures and the gel leaks into the body cavity, various health issues, such as autoimmune problems, may result.

Another problem is that both liquid and gel fillers may promote the growth of bacteria and mold in and around a breast implant. This mold and bacteria growth may contribute to other health problems.

Another problem is that humans are developing other types of cancers that may require implants. For example, testicular and penal cancers for men and other tissue and muscle cancers for both men and women. Accident patients and other non-cancer patients may require body contouring, such as for the gluteus, pectoral, calf, or other areas after an accident or surgery. In addition, many men and women are voluntarily requesting that the gluteus, pectoral, calf, lip and other areas not affected by cancer or other diseases be cosmetically enhanced with implants for body contouring. Current implant technologies are typically not suitable for reconstructive surgery for such body contouring.

There have been attempts to solve some of the problems associated with breast implants (and other types of implants). Enhanced silicone gel implants are being used. The enhanced silicone gels, which are thicker than previous gels, are more likely to stay in the implant in the event of a tear or rupture of the implant shell. These new silicone gels are called “cohesive silicone gels” and have already been used extensively outside the United States. However, these new cohesive silicone gels have not yet received FDA approval in the United States.

Another solution is the use of sustained mechanical force to induce tissue growth, which has been touted as a method to achieve modest growth and enlargement of the breasts. However, reported results have so far been inconclusive.

Another solution is new tissue engineering technology using stem cells derived from liposuctioned fat or other harvested cells that will be used to “re-grow” breast and other tissues. However, use of stem cells is currently controversial.

Another solution is to try and achieve breast enlargement through bio-manipulation of hormones or other chemical or biological substances that can be ingested or injected. However, the long term effects of these substances are still not known.

Thus, it is desirable to provide a new implant shell and filler apparatus that overcomes these and other problems associated with implants. There is a need for implants that will retain the feel of the tissue being replaced, and which will also substantially reduce or eliminate the probability of adverse biological reactions. Ideally, the implant will also not deflate.

### SUMMARY OF THE INVENTION

In accordance with preferred embodiments of the present invention, some of the problems associated with implants are overcome. An implant shell and filler apparatus is presented.

The implant shell includes a surface with a pre-determined material with a microbead filler. The implant shell and filler apparatus create a viable matrix for tissue growth that can also enhance the look, feel and touch of the implant in humans and animals. The implant shell and filler are made of an inert biocompatible material with non-permeable, permeable and/or semi-permeable characteristics. The implant shell and apparatus may also be used for animal tissues.

The foregoing and other features and advantages of preferred embodiments of the present invention will be more readily apparent from the following detailed description. The detailed description proceeds with references to the accompanying drawings.

### BRIEF DESCRIPTION OF THE DRAWINGS

Preferred embodiments of the present invention are described with reference to the following drawings, wherein:

FIG. 1 is a block diagram of a digital photograph illustrating plural predominantly spherical and/or rounded exemplary smooth microbeads;

FIG. 2 is a block diagram illustrating an exemplary microbead comprising a surface with plural indentations or holes;

FIG. 3 is a block diagram illustrating a microbead comprising a surface with woven material;

FIG. 4 is a block diagram illustrating a microbead comprising a surface with a perforated material;

FIG. 5 is a block diagram illustrating a portion of an implant shell including a woven material with plural microbeads visible;

FIG. 6 is a block diagram illustrating a side view of an implant shell of a human breast with plural microbeads visible;

FIG. 7 is a block diagram illustrating a top view of an implant shell of a human breast with plural microbeads visible;

FIG. 8 is a block diagram illustrating a side view of an implant shell of a human breast with plural microbeads and plural support channels visible;

FIG. 9 is a block diagram illustrating a top view of an implant shell of a human breast with plural microbeads and plural support channels visible;

FIG. 10 is a block diagram illustrating a top view of an implant shell of a human breast with plural microbeads and support ring visible;

FIG. 11 is a block diagram illustrating a side view of an implant shell of a human breast with plural microbeads and support ring visible; and

FIG. 12 is a block diagram illustrating a perspective side view of an implant shell of a human breast with plural microbeads and support ring visible.

### DETAILED DESCRIPTION OF PREFERRED EMBODIMENTS

The implant shell and filler apparatus are made of a biocompatible material with non-permeable, permeable and/or semi-permeable characteristics. The implant shell includes a pre-determined surface material with a microbead filler.

Implant Filler—Microbeads

In one embodiment, predominantly spherical and/or rounded microbeads are used to fill the implant shell. However, the present invention is not limited to such an embodiment and other shaped microbeads can also be used to practice the invention.

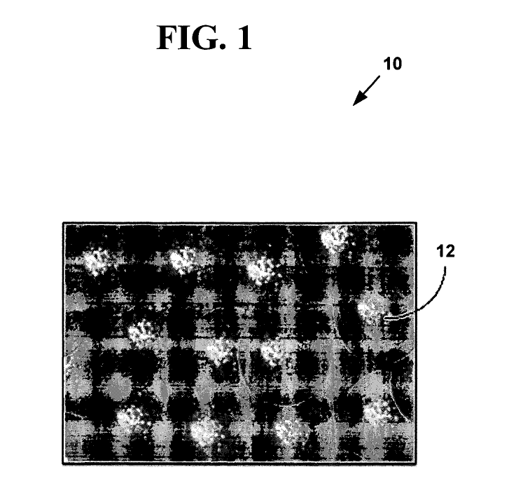

FIG. 1 is a block diagram 10 of a digital photograph of plural predominantly spherical and/or rounded smooth microbeads 12. In one embodiment, the plural smooth microbeads 12 include plural hollow or solid microbeads, or a combination thereof.

In one embodiment, the plural microbeads 12 comprise an inert biologically compatible material. In one embodiment, the plural microbeads comprise silicone, dacron, polystyrene, polypropylene, prolene, gortex, composite materials and other natural, biological and synthetic materials.

In another embodiment, the plural microbeads 12 also include non-biological inert materials that are coated with another biologically inert material. In another embodiment, the plural microbeads 12 also include biologically inert material coated with an identical or a different biologically inert material.

Microbeads made out of these described materials do not cause any autoimmune reactions in humans or animals and do not cause any additional diseases in humans or animals.

As is known in the art, microbeads have been used outside the medical arts for bean-bag chairs, pillows, toys and other devices. When microbeads are used to fill an apparatus, the apparatus is light, yet firm, and helps retain the shape of the container.

Microbeads have also been used in the medical arts for ultra-low non-specific binding and ultra-high binding entities for purification of proteins and nucleic acids, biological filters, biological markers in bioassays, and for other medical uses.

A preferred range of microbead sizes is from approximately 0.5 millimeters (mm) to approximately 1 mm in diameter. The microbeads are specifically sized to be large enough to be naturally trapped and filtered by (i.e., unable to enter) existing biological entities (e.g., lymph nodes, etc.) within a human or animal should the microbeads be released from a confining shell. Sizing the microbeads to allow natural biological filtering may prevent other problems such as heart attack or stroke should the microbeads accidentally enter a heart or brain of a human or animal. However, the present invention is not limited to such an embodiment and other sized microbeads can also be used to practice the invention.

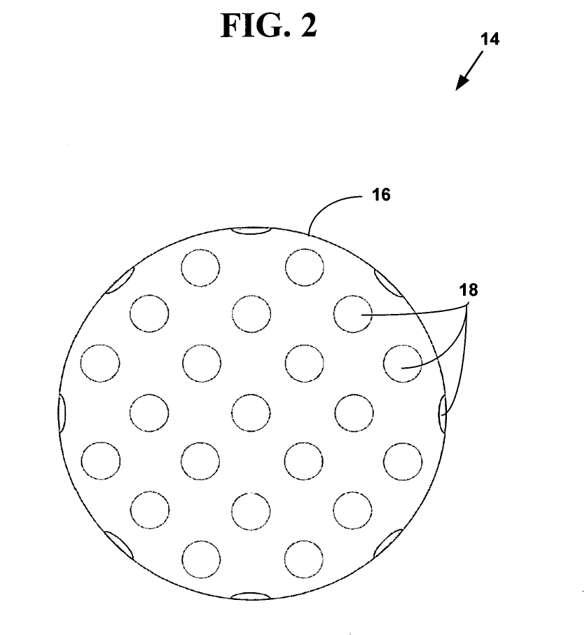

FIG. 2 is a block diagram 14 illustrating a microbead 16 comprising a surface with plural indentations and/or holes 18. The microbeads 16 may be solid, partially hollow or hollow.

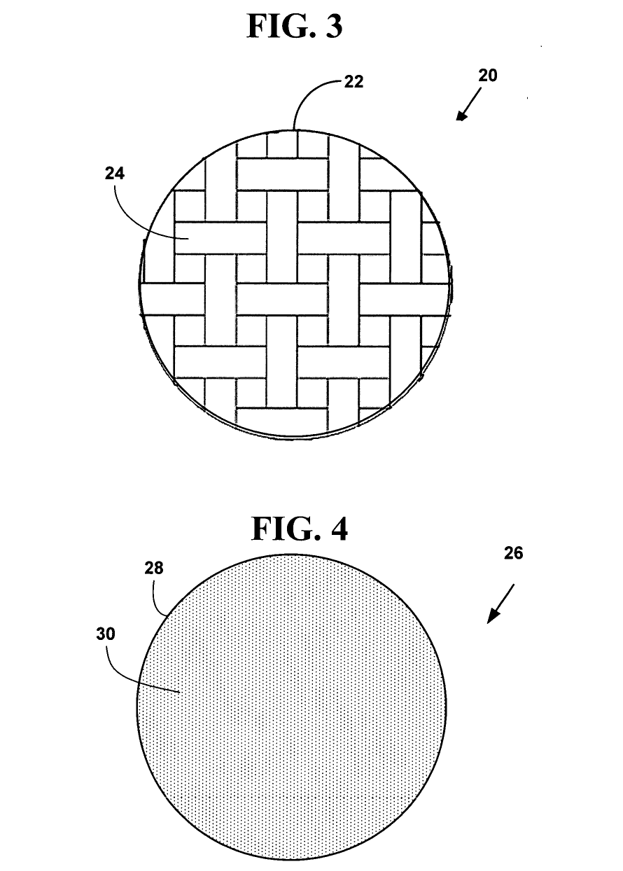

FIG. 3 is a block diagram 20 illustrating a microbead 22 comprising a surface with a woven material 24. The microbeads 22 may be solid, partially hollow or hollow.

FIG. 4 is a block diagram 26 illustrating a microbead 28 comprising a surface with a perforated material 30. The perforated surface may include a regular or an irregular pattern.

The indentations and/or holes 18, the woven material 24 and the perforated material 30 provide additional attachment points for tissue attachment (e.g., via tissue in-growth).

In various embodiments, the microbeads 12, 16, 22, 28 comprise a solid, hollow, or partially hollow interior with exterior surface of a smooth, perforated, indented or woven material.

In one embodiment, the microbeads 12, 16, 22, 28 are all identical in size. In another embodiment, the microbeads 12, 16, 22, 28 are variable, but similar in size. In another embodiment, the microbeads 12, 16, 22, 28 are not similar in size and include plural different sizes. In another embodiment, the microbeads 12, 16, 22, 28 vary in shape.

In one embodiment, variations in size and shape among the microbeads enhance the movement of the microbeads within the implant shell. In another embodiment, microbeads of the same size and shape promote a most natural movement within the implant shell. Movement of the microbeads within the implant shell contributes to the natural look and feel of the implant.

However, the present invention is not limited to such embodiments and other types, variations and combinations of microbeads can also be used to practice the invention.

In one embodiment, materials between the microbeads 12, 16, 22, 28 include air, saline, interstitial fluid or some other acceptable fluid. The microbeads 12, 16, 22, 28 shift within the implant shell when touched, resulting in a soft, flexible, natural feel.

The microbeads 12, 16, 22, 28 are also light in weight so a breast or other body part will not be weighed down and will have a natural look and feel when touched.

This has a positive effect on the musculature of the patient as well as the patient's psychological well-being.

Implant Shell

In one embodiment, the implant shell comprises a pre-determined surface with a woven material. In another embodiment, the implant shell comprises a pre-determined surface with a non-woven material. In another embodiment, the implant shell includes a perforated material. In another embodiment, the implant shell comprises existing implant shell known and the art filled with microbeads 12, 16, 22, 28.

The implant shell made from the woven or perforated materials includes permeable characteristics that allow for body fluids to enter and exit the implant. In the case of microbeads that are made of holes 18, woven 24 or perforated material 30, the same permeable characteristics allow for body fluid to enter and exit the microbeads themselves. Such permeability allows for tissue in-growth. Among other things, tissue in-growth reduces encapsulation and has general and specific microbiological and biochemical advantages in many patients.

Woven materials of numerous types of compositions have already been used in other internal medical applications and include dacron and a host of other polymer meshes (e.g., silicon, polypropylene, prolene, gortex, composite materials, etc.). As in human medical and veterinarian applications, it is preferable to use an antibiotic soak before inserting the woven fabric of the fill shell into the body.

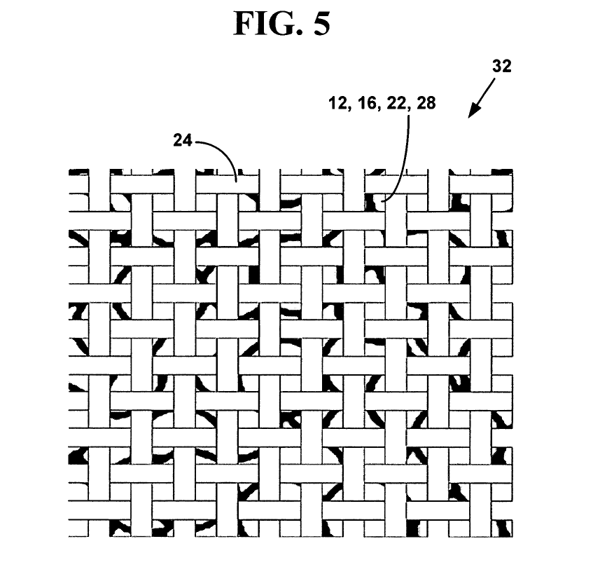

FIG. 5 is a block diagram 32 illustrating a portion of an implant shell including a woven material 24 with plural microbeads 12, 16, 22, 28 visible.

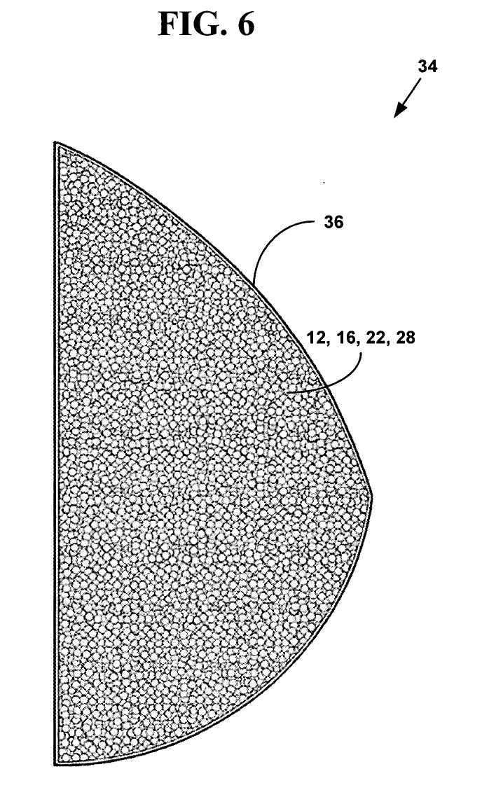

FIG. 6 is a block diagram 34 illustrating a side view of an implant shell 36 of a human breast with plural microbeads 12, 16, 22, 28 visible.

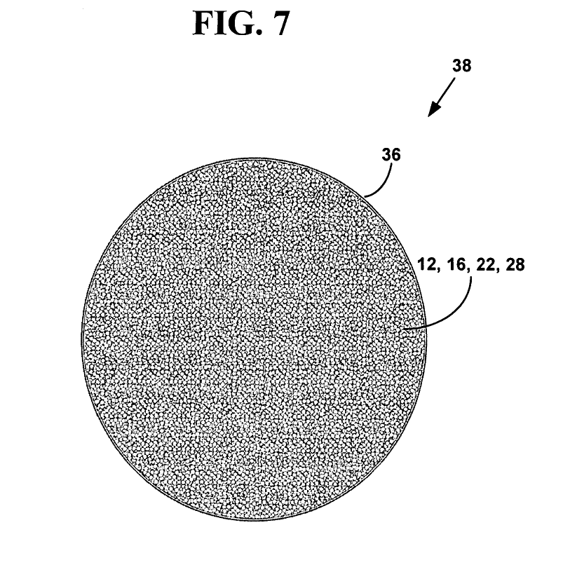

FIG. 7 is a block diagram 38 illustrating a top view of an implant shell 36 of a human breast with plural microbeads 12, 16, 22, 28 visible.

In another embodiment, the implant shell may further include one or more support channels. The support channels help support and shape the implant shell and make it more durable and less subject to collapse or contraction. The support channels may be solid, partially hollow, hollow or perforated. If the support channels are hollow, partially hollow or perforated, they allow fluid movement through the channels. In one embodiment, the support channels include a woven material identical to that used for implant shell 36. In another embodiment, the support channels include a surface material not identical to that used for the implant shell 36.

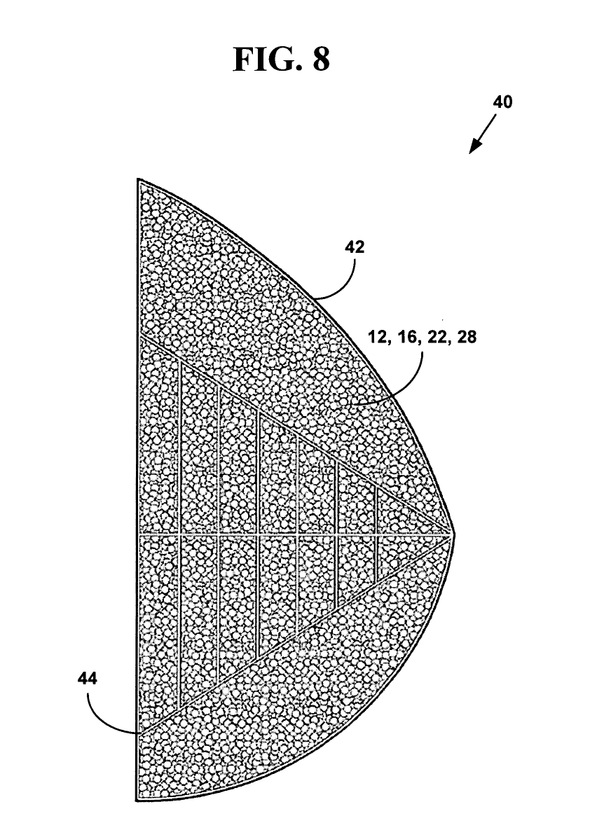

FIG. 8 is a block diagram 40 illustrating a side view of an implant shell 42 of a human breast with plural microbeads 12, 16, 22, 28 and plural support strands/channels 44 visible.

FIG. 8 illustrates an implant shell 42 with plural support strands/channels 44 in the shape of a triangle. However, the present invention is not limited to such a shape for the plural support strands/channels 44 and other regular (e.g., square, rectangle, trapezoid, oval, etc.) and irregular shapes may be used to practice the invention.

In one embodiment, the plural support channels are packed with a material denser than the microbeads 12, 16, 22, 28. In such an embodiment, the plural support channels with the denser material provide a core of material to improve the shape of the implant, making it look and feel more like a natural breast.

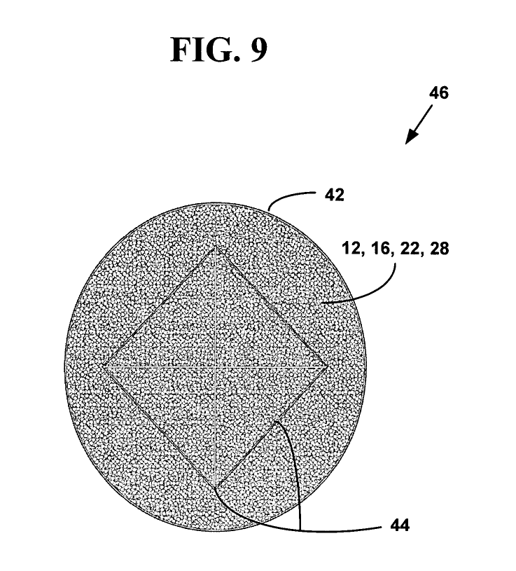

FIG. 9 is a block diagram 46 illustrating a top view of an implant shell 42 of a human breast with plural microbeads 12, 16, 22, 28 and plural support strands/channels 44 visible.

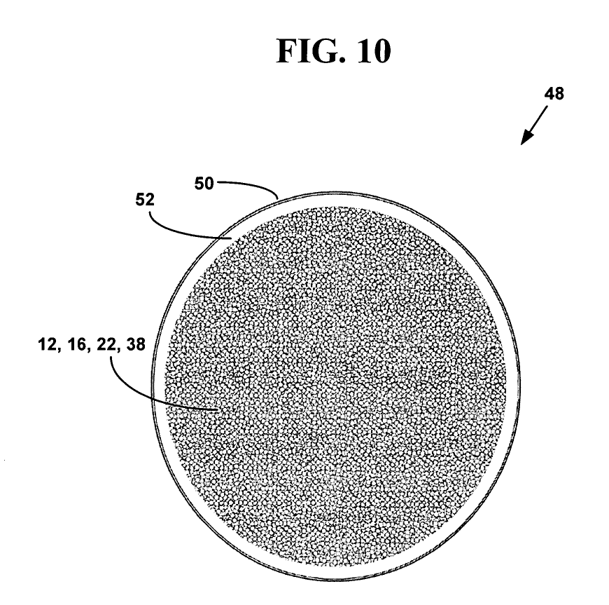

FIG. 10 is a block diagram 48 illustrating a top view of an implant shell 50 of a human breast with plural microbeads 12, 16, 22, 28 and support ring 52 visible.

In another embodiment, the implant shell 50 may further comprise a hollow, partially hollow, perforated or solid support ring 52. Such support ring adds additional support to the implant shell 50. If the support ring is hollow, it may be filed with an appropriate material (e.g., silicone, saline, etc. solutions, microbeads 12, 16, 22, 28, silicone gels, other gels, etc.). If the support ring is solid, it may comprise identical materials as the implant shell 50, or may comprise a material different from the implant shell 50. In one embodiment, the support ring 52 includes a woven or perforated material.

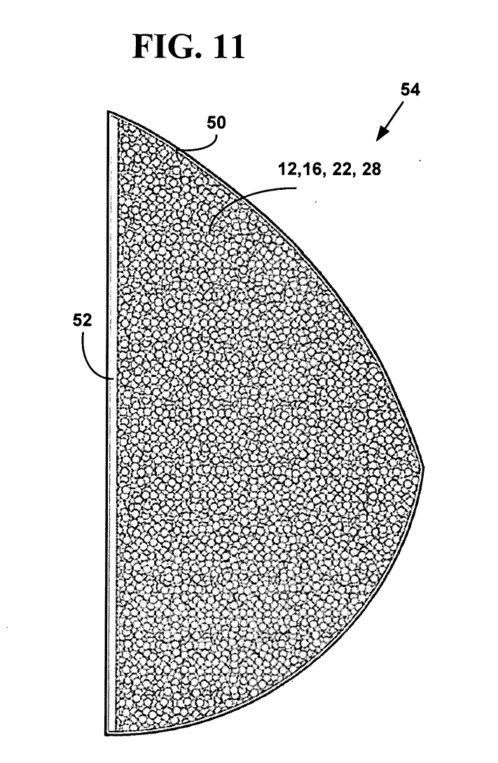

FIG. 11 is a block diagram 54 illustrating a side view of an implant shell 50 of a human breast with plural microbeads 12, 16, 22, 28 and support ring 52 visible.

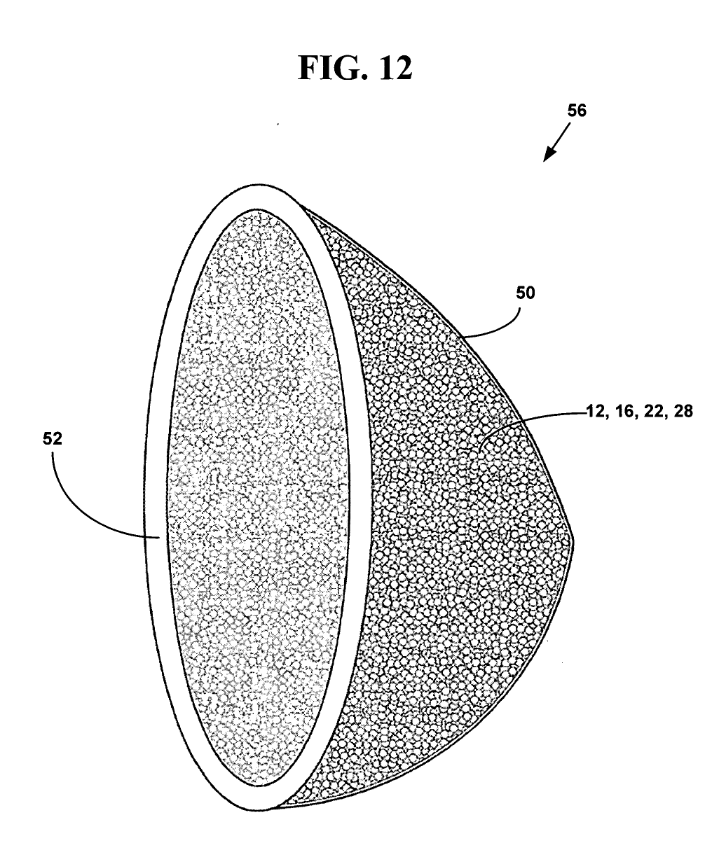

FIG. 12 is a block diagram 56 illustrating a perspective side view of an implant shell 50 of a human breast with plural microbeads 12, 16, 22, 28 and support ring 52 visible.

The implant shell and filler apparatus described herein are made of an inert biocompatible material with permeable and/or semi-permeable and/or non-permeable characteristics. The implant shell and filler apparatus can replace, augment or otherwise alter breasts, testicles and other human and animal body parts and tissue. The implant fill and shell apparatus can also be used for needed or desired human body contouring, such as for the gluteus, pectoral and calf areas. Additionally, the implant shell and fill apparatus can be used for urological applications including the oval carving block, testicular implant, malleable penile implant, clitoral implant, labia implant, vesical conformer, periurethral constrictor and vaginal stents. Further, maxillofacial implants, nostril retainers and all other custom implants benefit from application of this invention. The invention can also be used for lumpectomy implants, mastectomy implants, and/or cosmetic implants.

It should be understood that the specific components and apparatus described herein are not related or limited to any particular type apparatus unless indicated otherwise. Various types of general purpose or specialized apparatus may be used with or perform operations in accordance with the teachings described herein.

In view of the wide variety of embodiments to which the principles of the present invention can be applied, it should be understood that the illustrated embodiments are exemplary only, and should not be taken as limiting the scope of the present invention. For example, the apparatus described may include more or fewer elements.

The claims should not be read as limited to the described order or elements unless stated to that effect. In addition, use of the term “means” in any claim is intended to invoke 35 U.S.C. §112, paragraph 6, and any claim without the word “means” is not so intended.

Therefore, all embodiments that come within the scope and spirit of the following claims and equivalents thereto are claimed as the invention.

I claim:

1. A prosthetic implant, comprising, in combination:
an outer shell including a pre-determined surface material having a cavity therein, the outer shell being specifically sized and shaped for augmenting, reshaping or replacing human body parts; and
a filler for filling the outer shell having a plurality of microbeads contained in the cavity of the outer shell.

2. The prosthetic implant of claim 1 wherein the pre-determined surface material of the outer shell includes a woven, perforated or indented material.

3. The prosthetic implant of claim 2 wherein the woven or perforated or indented material allows fluid movement into and out of the prosthetic implant thereby promoting tissue in-growth around the prosthetic implant and reducing encapsulation in the prosthetic implant.

4. The prosthetic implant of claim 1 wherein the outer shell comprises a silicone, dacron, polystyrene, polypropylene, prolene, gortex, or composite materials.

5. The prosthetic implant of claim 1 wherein the outer shell includes a non-permeable, permeable or semi-permeable biological inert material.

6. The prosthetic implant of claim 1 wherein the outer shell is coated with a biological inert material.

7. The prosthetic implant of claim 1 wherein the outer shell further includes one or more support channels.

8. The prosthetic implant of claim 7 wherein the one or more support channels comprise a triangle shape.

9. The prosthetic implant of claim 7 wherein the triangle shape includes an apex of a triangle at portion of prosthetic implant corresponding to a middle portion of a nipple of human breast.

10. The prosthetic implant of claim 7 wherein the one or more support channels are hollow, partially hollow or solid.

11. The prosthetic implant of claim 1 wherein the outer shell further includes a support ring.

12. The prosthetic implant of claim 11 wherein the support ring includes a hollow, partially hollow or solid support ring.

13. The prosthetic implant of claim 12 wherein the hollow support ring is filled with a plurality of microbeads.

14. The prosthetic implant of claim 1 wherein the plurality of microbeads comprise silicone, dacron, polystyrene, polypropylene, prolene, gortex or composite material.

15. The prosthetic implant of claim 1 wherein the plurality of microbeads comprise a plurality of hollow, partially hollow or solid microbeads.

16. The prosthetic implant of claim 1 wherein the plurality of microbeads comprise a plurality of indentations or holes on a surface of the plurality of microbeads.

17. The prosthetic implant of claim 1 wherein the plurality of microbeads comprise a plurality of microbeads with a woven surface or a perforated surface.

18. The prosthetic implant of claim 1 wherein the prosthetic implant replaces, augments or alters human breast or human testicle tissue.

19. The prosthetic implant of claim 1 wherein the prosthetic implant provides human tissue contouring.

20. A prosthetic implant, comprising, in combination:
a containment means of pre-determined surface material having a cavity therein, the containment means being specifically sized and shaped for augmenting, reshaping or replacing human body parts; and
a filler means for filling the containment means having a plurality of microbeads contained in the cavity therein.

21. The prosthetic implant of claim 20 wherein the prosthetic implant replaces, augments or alters human breast or human testicle tissue or provides other human tissue contouring.

22. The prosthetic implant of claim 20 wherein the pre-determined surface includes a permeable or semi-permeable material which allows the movement of body fluids into and out of the prosthetic implant thereby promoting tissue in-growth around the prosthetic implant and reducing encapsulation in the prosthetic implant.

23. The prosthetic implant of claim 20 wherein the plurality of microbeads includes a plurality of indentations/holes or a woven material on a surface of the plurality of microbeads.

24. A prosthetic implant, comprising, in combination:
an outer shell of a permeable or semi-permeable woven surface material having a cavity therein, the outer shell being specifically sized and shaped for augmenting, reshaping or replacing human or animal body parts or human or animal tissues; and
a filler for filling the outer shell having a plurality of microbeads contained in the cavity of the outer shell, wherein the plurality of microbeads having a surface with a plurality of indentations or holes or a woven surface material or a solid surface.
